(19) United States
(12) Patent Application Publication
(10) Pub. No.: US 2008/0255495 A1
(43) Pub. Date: Oct. 16, 2008

(54) Tampon with flexible panels

(76) Inventors: David J. Chase, Somerville, NJ (US); Erin Danyi, Nicholasville, KY (US); Tara Glasgow, New Hope, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/051,562

(22) Filed: Mar. 19, 2008

Related U.S. Application Data
(63) Continuation of application No. 10/848,257, filed on May 14, 2004.

(51) Int. Cl. A61F 13/20

(52) U.S. Cl. 604/11; 604/385.18; 604/385.17

(57) ABSTRACT

An intravaginal device has a fluid storage element having a longitudinal axis and is in fluid communication with at least one flexible panel.

### CROSS-REFERENCE TO RELATED APPLICATIONS

This invention is related to the following copending applications: “Intravaginal Device with Fluid Acquisition Plates” (U.S. Ser. No. 60/572,054; Atty Docket No. PPC-5073), “Intravaginal Device with Fluid Acquisition Plates and Method of Making” (U.S. Ser. No. 60/572,055; Atty Docket No. PPC-5072), “Fluid Management Device with Fluid Transport Element for use within a Body” (U.S. Ser. No. 10/847,951; Atty Docket No. PPC-5071), “Method of Using Intravaginal Device with Fluid Transport Plates” (U.S. Ser. No. 10/848,347; Atty Docket No. PPC-5076), “Method of Using an Intravaginal Device with Fluid Transport Plates” (U.S. Ser. No. 10/848,208; Atty Docket No. PPC-5075), and “Intravaginal Device with Fluid Acquisition Plates” (U.S. Ser. No. 10/847,952; Atty Docket No. PPC-5070), the content of each of which is incorporated herein.

### FIELD OF THE INVENTION

The present invention relates to devices (e.g., intravaginal tampons) for capturing and storing bodily fluid. More particularly, the invention relates to an improved tampon that has a compressed core portion and at least one flexible panel extending therefrom for improved fluid movement during use.

### BACKGROUND OF THE INVENTION

Devices for capturing and storing bodily fluid intravaginally are commercially available and known in the literature. Intravaginal tampons are the most common example of such devices. Commercially available tampons are generally compressed cylindrical masses of absorbent fibers that may be over-wrapped with an absorbent or nonabsorbent cover layer.

The tampon is inserted into the human vagina and retained there for a time for the purpose of capturing and storing intravaginal bodily fluids, most commonly menstrual fluid. As intravaginal bodily fluid contacts the tampon, it should be absorbed and retained by the absorbent material of the tampon. After a time, the tampon and its retained fluid is removed and disposed, and if necessary, another tampon is inserted.

A drawback often encountered with commercially available tampons is the tendency toward premature failure, which may be defined as bodily fluid leakage from the vagina while the tampon is in place, and before the tampon is completely saturated with the bodily fluid. The patent art typically describes a problem believed to occur that an unexpanded, compressed tampon is unable to immediately absorb fluid. Therefore, it presumes that premature leakage may occur when bodily fluid contacts a portion of the compressed tampon, and the fluid is not readily absorbed. The bodily fluid may bypass the tampon.

To overcome this problem of premature leakage, extra elements have been incorporated into a basic tampon to try to direct and control the flow of fluid toward the absorbent core.

For example, U.S. Pat. No. 4,212,301 (Johnson) discloses a unitary constructed digital tampon having a lower portion compressed preferably in the radial direction to form a rigid, rod-like element, which provides a central rigidified elongated core and an upper portion left substantially uncompressed. After insertion, the uncompressed portion may be manipulated to contact the vaginal wall to provide an immediate seal against side leakage. The uncompressed portion allows for high absorbent capacity immediately upon insertion. While this tampon may allow for a certain amount of protection from bypass leakage, the uncompressed portion may become saturated before the compressed portion has a chance to expand and become absorbent.

U.S. Pat. No. 6,358,235 (Osborn et al.) discloses a “hollow” bag-like tampon that may have an interior projection made from highly compressed absorbent material. The interior projection is preferably attached to the inside surface of the head of the tampon. The hollow tampon portion may include at least one pleat in the absorbent outer surface and is soft and conformable. The tampon is not pre-compressed to the point where the fibers temporarily “set” and re-expand upon the absorption of fluid. The absorbent portions of the tampon can saturate locally, which leads to bypass leakage.

U.S. Pat. No. 6,177,608 (Weinstrauch) discloses a tampon having nonwoven barrier strips which are outwardly spreadable from the tampon surface to reliably close the free spaces believed to exist within a vaginal cavity. The nonwoven barrier strips extend about the tampon in a circumferential direction at the surface or in a helical configuration about the tampon and purportedly conduct menstrual fluid toward the tampon surface. The nonwoven barrier strips are attached to the cover by means of gluing, heat sealing, needle punching, embossing or the like and form pleats. The nonwoven barrier strips are attached to the tampon blank and the blank is embossed, forming grooves extending in a longitudinal direction. While this tampon purports to direct fluid to the core, it attempts to achieve this by forming pockets of absorbent nonwoven fabric. In order to function, it appears that these pockets would have to be opened during use to allow fluid to enter. However, based upon current understandings of vaginal pressures, it is not understood how the described structure could form such an opened volume.

U.S. Pat. No. 6,206,867 (Osborn) suggests that a desirable tampon has at least a portion of which is dry expanding to cover a significant portion of the vaginal interior immediately upon deployment. To address this desire, it discloses a tampon having a compressed central absorbent core having at least one flexible panel attached along a portion of the side surface of the core. The flexible panel appears to provide the “dry-expanding” function, and it extends outwardly from the core away from the point of attachment. The flexible panel contacts the inner surfaces of the vagina when the tampon is in place and purportedly directs fluid toward the absorbent core. The flexible panel is typically attached to the pledget prior to compression of the pledget to form the absorbent core and remains in an uncompressed state.

U.S. Pat. No. 5,817,077 (Foley et al.) discloses a method of preserving natural moisture of vaginal epithelial tissue while a using a tampon where the tampon has an initial capillary suction pressure at the outer surface of less than about 40 mm Hg. This allows the tampon to absorb vaginal secretions without substantially drying the vaginal epithelial tissue. The multiple cover layers can be used to increase the thickness of the cover material. While this represents a significant advancement in the art, this invention does not address by-pass leakage.

Additionally, U.S. Pat. No. 5,545,155 (Hseih et al.) discloses an external absorbent article that has a set of plates separated by spacer elements. The plates may be treated to affect wettability so that fluid will flow easily across the surface. Extending through the upper plate is a plurality of openings, which allow fluid to flow with little restriction into the space between the upper and lower plates. When the fluid flows downward in the z-direction from the upper plate to the lower plate, it will then flow laterally in the x- and y-directions. Therefore, this external absorbent article can contain fluid gushes, but it does not appear to address the problems relating in particular to intravaginal devices, such as a tampon.

While the prior art is replete with examples of sanitary protection articles that capture bodily fluids both externally and intravaginally, these examples do not overcome the problem of premature failure often identified as by-pass leakage that commonly occurs while using internal sanitary protection devices. Many solutions to this problem have involved increasing the rate of expansion of a highly compressed absorbent article.

### SUMMARY OF THE INVENTION

It has been discovered that the problem of premature failure can be addressed in a surprising and different way. The present invention is not dependent on the expansion of the compressed absorbent, but rather, it directs the fluid by the use of inter-plate capillary action. In our invention, we minimize local saturation of the fluid storage element. Our invention also is effective for handling highly viscous menstrual fluid.

In one aspect of the invention a packaged intravaginal device includes a fluid storage element having a longitudinal axis and an outer surface, a flexible panel in fluid communication with the fluid storage element, and a packaging element. The flexible panel has a distal portion capable of extending radially outward from the fluid storage element. It is also bendable about an axis substantially parallel to the longitudinal axis of the fluid storage element. The packaging element substantially encloses the intravaginal device with at least a portion of a major surface of the flexible panel in contact with at least a portion of the side surface of the fluid storage element.

In another aspect, an elongate, packaged vaginal tampon has a length oriented in an axial direction. It includes a fluid storage element, at least one flexible panel, and a packaging element. The fluid storage element is constructed from an absorbent material compressed to a self-sustaining form. It has a first end corresponding to an insertion end of the tampon, a second end disposed opposite the first end, a side surface extending between the first end and the second end, and a perimeter, measured in a plane perpendicular to the axial direction. The fluid storage element is extendable radially outward from the fluid storage element. The packaging element substantially encloses the tampon with at least a portion of a major surface of the flexible panel in contact with at least a portion of the side surface of the fluid storage element.

Other aspects and features of the present invention will become apparent in those ordinarily skilled in the art upon review of the following description of specific embodiments of the invention in conjunction with the accompanying drawings.

### BRIEF DESCRIPTION OF THE DRAWING

FIG. 1 is a side elevation of a packaged tampon according to the present invention.

FIG. 2 is a side elevation of a tampon of the present invention with a compressed absorbent core and flexible panels extending therefrom.

FIG. 3 is a transverse cross-section of an example of the tampon of FIG. 1 upon insertion into a vagina having one panel extending into the vagina.

FIG. 4 is a transverse cross-section of an example of the tampon of FIG. 1 upon insertion into a vagina without any panels extending into the vagina.

FIG. 5a shows a side elevation of an alternate embodiment of the present invention in which a cover material is bonded to itself in the form of a bag to form flexible panels in fluid communication with a fluid storage element.

FIG. 5b shows a cross-sectional view of the device in FIG. 5a taken along line 5b-5b.

FIG. 6 shows a side elevation of an embodiment of the present invention in which the flexible panels encase the fluid storage element and are bonded at the withdrawal end to the withdrawal string.

FIG. 7 shows a side elevation of an embodiment of the present invention in which the flexible panels encase the fluid storage element and are bonded to the base of the fluid storage element.

FIG. 8 is a side elevation of a tampon of the present invention prior to the compression of an intermediate structure.

FIG. 9 is a top view showing one manner in which the intermediate structure of the tampon of the present invention may be compressed.

FIG. 10 is a partially cut-away side elevation of a tampon of the present invention within an applicator.

FIG. 11 is a transverse cross-section of a tampon according to the present invention having flexible panels wrapped around the fluid storage element.

FIG. 12 is a transverse cross-section of a tampon according to the present invention having flexible panels folded about the fluid storage element.

### DETAILED DESCRIPTION OF THE PREFERRED EMBODIMENTS

As used herein in the Specification and the Claims, the term “bodily fluid” and variants thereof mean bodily exudates, especially liquids that are produced by, secreted by, emanate from, and/or discharged from a human body.

As used herein in the Specification and the Claims, the term “fluids” and variants thereof relate to liquids, and especially bodily fluids.

As used herein in the Specification and the Claims, the term “sheet” and variants thereof relates to a portion of something that is thin in comparison to its length and breadth.

As used herein in the Specification and the Claims, the term “porous medium” and variants thereof relates to a connected 3-dimensional solid matrix with a highly ramified network of pores and pore throats in which fluids may flow.

As used herein in the Specification and the Claims, the term “in fluid communication” and variants thereof relate to elements that are arranged and configured to allow fluid to move therebetween. The fluid movement may be by interfiber capillary movement, intrafiber capillary movement, osmotic pressure, inter-plate capillary action, mechanical channeling, and the like.

As used herein in the Specification and the Claims, the term “coupled” and variants thereof relate to the relationship between two portions of an integral structure that are either portions of the same material (e.g., two portions of a folded sheet) or are materials that are joined together (e.g., two separate sheets that are bonded together).

As used herein in the Specification and the Claims, the term “fluid-permeable cover” and variants thereof relates to materials that cover or enclose surfaces of the device and reduce the ability of portions (e.g., fibers and the like) from becoming separated of the device and left behind upon removal. The term and variants thereof include, without limitation, sheet-like materials, such as apertured films and woven and non-woven fibrous webs, surface treatments, such as coatings or cover layers of integrating materials, such as binders and thermobondable fibers, and the like.

This invention relates to devices (e.g., intravaginal tampons) for capturing and storing bodily fluid. More particularly, the invention relates to an improved tampon which has a compressed core portion and at least one flexible panel for improved coverage of the interior of the vaginal cavity and direction of acquired fluid to the tampon core.

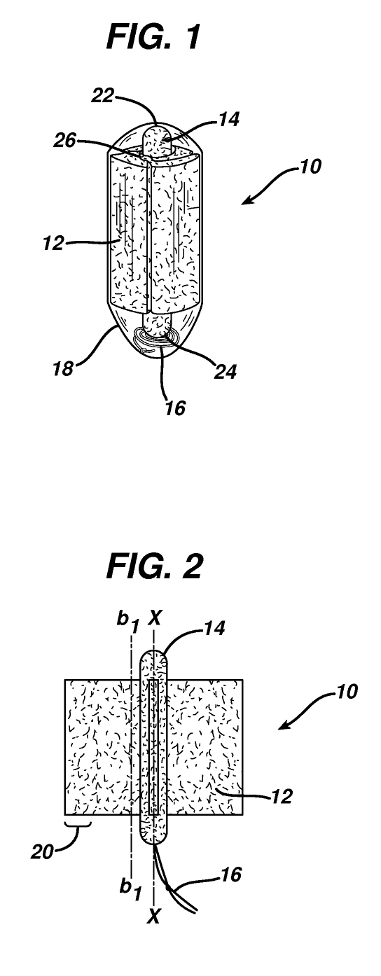

Referring to FIG. 1, one embodiment of this invention provides a packaged tampon 10 having at least one flexible panel 12 in fluid communication with a fluid storage element 14 (FIG. 1 shows a plurality of flexible panels 12 located about and extending from sides of the fluid storage element 14). The tampon may also include a withdrawal mechanism, such as a string 16. The flexible panels 12 are shown wrapped around the fluid storage element 14. The panels are maintained in this configuration by hygienic overwrap 18. However, as shown in FIG. 2, a distal portion 20 of the flexible panels 12 may extend radially away from the fluid storage element 14 during use.

The flexible panels can be made of almost any hydrophobic or hydrophilic material, preferably a sheet-like web. For example, the panel(s) 12 may be constructed from a wide variety of liquid-absorbing or liquid-transporting materials commonly used in absorbent articles such as rayon, cotton, or comminuted wood pulp which is generally referred to as airfelt. Examples of other suitable absorbent materials include creped cellulose wadding; meltblown polymers including conform; chemically stiffened, modified or cross-linked cellulosic fibers; synthetic fibers such as crimped polyester fibers; peat moss; tissue including tissue wraps and tissue laminates; or any equivalent material or combinations of materials, or mixtures of these. Preferred materials comprise cotton, rayon (including tri-lobal and conventional rayon fibers, and needle punched rayon), folded tissues, woven materials, nonwoven webs, synthetic and/or natural fibers. The panel(s) 12 and any component thereof may comprise a single material or a combination of materials.

The thickness of each panel is not critical. However, it can preferably be selected from the range of from about 0.005 to about 0.050 inch. Preferably, the materials of construction and the thickness of the panels are designed to be sufficiently stiff and/or resistant to wet collapse when exposed to fluid.

In particular, materials useful for forming the flexible panel may have properties such as thermobondability to provide means to incorporate it into the intravaginal device. A representative, non-limiting list of useful materials includes polyolefins, such as polypropylene and polyethylene; polyolefin copolymers, such as ethylenevinyl acetate (“EVA”), ethylene-propylene, ethyleneacrylates, and ethylene-acrylic acid and salts thereof, halogenated polymers; polyesters and polyester copolymers; polyamides and polyamide copolymers; polyurethanes and polyurethane copolymers; polystyrenes and polystyrene copolymers; and the like.

The flexible panel 12 should be strong enough to prevent rupturing during handling, insertion, and removal and to withstand vaginal pressures during use.

It is preferable that the flexible panel(s) 12 are sufficiently wettable by the bodily fluids that the intravaginal device 10 is intended to collect (this results largely from a correlation of the surface energy of the panel surface and the bodily fluid(s)). Thus, the bodily fluid will easily wet the panel, and a driving mechanism can divert fluid toward the fluid storage element 14. In particularly preferred embodiments, this driving mechanism is provided though the use of capillary channel fibers, an osmotic driving force, a hydrophilicity gradient, a capillary driving force, or some combination of these.

Surface treatments can be used to modify the surface energy of the panel(s) 12. In a preferred embodiment a surfactant is applied to increase the wettability of the panel(s) 12. This will increase the rate at which the bodily fluids are drawn into and transported by a flexible panel. The surfactant can be applied uniformly, or it can be applied with varying coating weights in different regions.

A useful measure to determine the wettability of a panel material is its contact angle with 1.0% saline. Preferably, the contact angle with 1.0% saline is less than about 90 degrees.

In order to accomplish this, the materials of panel(s) can be chosen from those materials that are known in the art to have low energy surfaces. It is also possible and useful to coat materials with high-energy surfaces with a surface additive, such as a non-ionic surfactant (e.g., ethoxylates), a diol, or mixtures thereof, in order to increase their wettability by bodily fluids. Such additives are well known in the art, and examples include those described in Yang et al., US App. No. 2002-0123731-A1, and U.S. Pat. No. 6,570,055. Other means of increasing wettability can also be used, such as blending in hydrophilic fibers, etc.

The flexible panel can be of any flexibility as long as the material is able to transport fluid to the fluid storage element while the device is in use. It is also preferable that the flexible panel be sufficiently flexible to provide the user with comfort while inserting, wearing and removing the device.

The panels are bendable about an infinite number of bending axes (b₁₋ᵢ-b₁₋ᵢ) that are substantially parallel to the longitudinal axis (X-X) of the product, which longitudinal axis extends through the insertion end 22 and withdrawal end 24. These bending axes allow the panels to wrap around the product, either partially or completely.

The flexible panel 12 is in fluid communication with the fluid storage element 14 and directs fluid from the vagina to the storage element 14. Generally, fluid will be directed from each flexible panel 12 to a particular region of the fluid storage element associated with that flexible panel. Thus, if the device has only one flexible panel 12, the fluid will contact the fluid storage element in one interface 26.

Therefore, additional flexible panels 12 directing fluid to additional locations of the fluid storage element 14 will improve the efficient usage of the fluid storage element 14. For example, four evenly spaced flexible panels 12 allow fluid to be directed to each quarter of the fluid storage element 14 surface as shown in FIG. 4, discussed in greater detail, below. Five or more elements would provide even more direct access. This can allow the fluid to contact the fluid storage element 14 uniformly and help to prevent or reduce local saturation of the fluid storage element 14. Each additional flexible panel 12 can direct fluid to additional interface locations 26 of the fluid storage element 14.

Enlarging the area of the interface 26 between the flexible panel 12 and fluid storage element 14 can also help to maximize the fluid communication. For example, elongating the interface by increasing the length of the flexible panel 12 allows more fluid to flow into the fluid storage element 14.

While the above description provides for direct fluid communication between a flexible panel 12 and the fluid storage element 14, direct fluid contact is not necessary. There can be fluid communication through an intermediate element, such as a porous medium (e.g., a foam or fibrous structure), a hollow tube, and the like. Thus, the flexible panel(s) 12 may be indirectly secured to the other element by affixing the element to intermediate member(s) which in turn are affixed to the other element; and configurations in which one element is integral with another element; i.e., one element is essentially part of the other element.

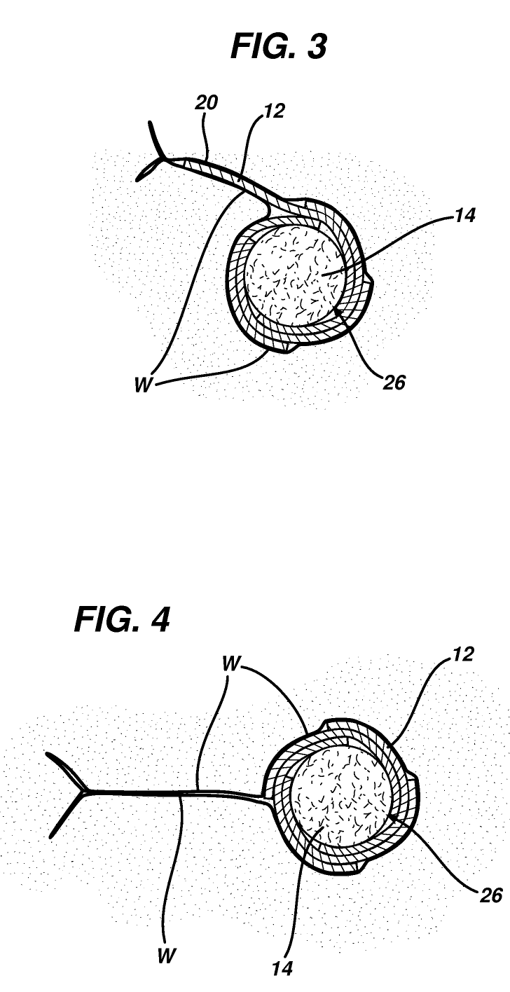

The flexible panel 12 may be formed to extend from the surface of the fluid storage element 14 as in FIGS. 2-3. It can be made in any convenient shape, including semicircular, triangular, square, hourglass etc.

During use, flexible panel(s) 12 can take on many configurations within the vagina. For example, a distal portion 20 of the flexible panel 12 may extend into the vagina away from the fluid storage element 14, as shown in FIG. 3. Alternatively, and the flexible panel(s) 12 may remain wound about the fluid storage element 14, contacting the vaginal wall “W” only through a major surface 28 (FIG. 4).

The flexible panels 12 may be joined to the fluid storage element 14 by any variety of means. For example, the flexible panels 12 may be joined to the fluid storage element 14 using any suitable adhesive. Such adhesive may extend continuously along the length of attachment or it may be applied in a “dotted” fashion at discrete intervals. Alternatively, the flexible panels 12 may be joined to the fluid storage element 14 by stitching. Such stitching may use cotton or rayon thread. Other attachment mechanisms include thermally bonding (for example where the tampon core and panels have thermally bonded fibers or other thermally bonding materials incorporated therein), fusion bonding, or any other suitable means known in the art for joining such materials.

The flexible panel(s) 12 can be attached at the sides, insertion end 22, and/or withdrawal end 24 of the intravaginal device. Additionally, the flexible panel(s) 12 may be attached to themselves and not to the storage element as in a flexible panels bag type covering of the storage element. The flexible panels could also be attached to the withdrawal string. These and other means of attachment are disclosed in the commonly-assigned, copending patent applications entitled “Intravaginal Device with Fluid Acquisition Plates” (U.S. Ser. No. 60/572,054; Atty Docket No. PPC-5073), “Intravaginal Device with Fluid Acquisition Plates and Method of Making” (U.S. Ser. No. 60/572,055; Atty Docket No. PPC-5072), the contents of which are herein incorporated by reference.

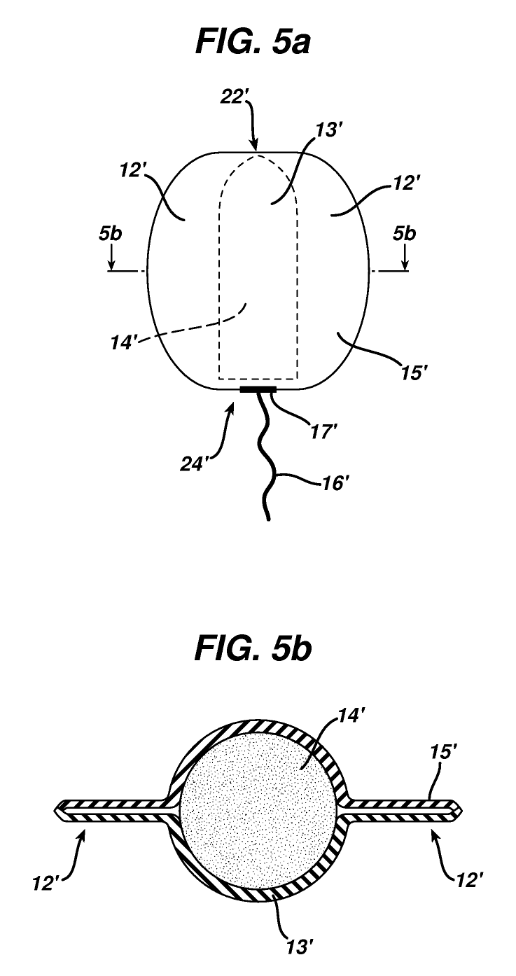

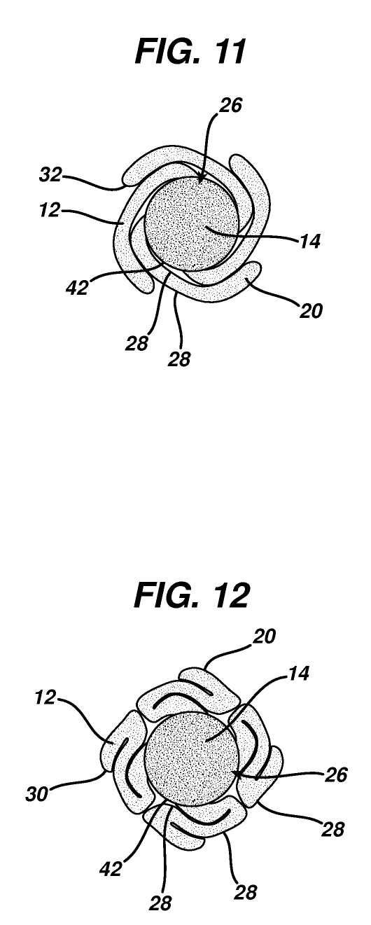

In an embodiment shown in FIGS. 5a-b, the sheet used to form the flexible panel 12′ substantially encases the fluid storage element 14′ (shown as a tampon), forming a bag- or sack-type structure 13′ having at least one pleat 15′. In one embodiment, the sheet that forms the flexible panel is draped over the insertion end 22′ of the tampon with the ends of the sheet brought together about the withdrawal end 24′ and then sealed to itself. The excess fluid transport material about the sides of the fluid storage element forms folds or pleats 15′. The pleats can then be twisted around the tampon similarly as shown in FIG. 11, discussed in greater detail, below.

Upon insertion, which can be digitally or through the use of an applicator, as previously mentioned the transport element may extend into the vagina away from the fluid storage element.

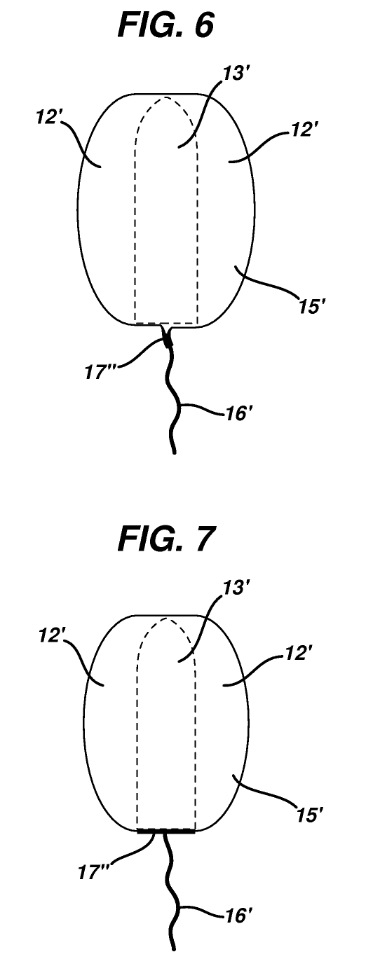

Other embodiments similar to that shown in FIGS. 5a-b are possible. For example, the flexible panel 12′ may be sealed to the withdrawal string 16′ (FIG. 6) or alternately, to the withdrawal end 24′ of the fluid storage element 14′ (FIG. 7). In all of these embodiments, the flexible panel encases the fluid storage element but does not affect the fluid storage element. For example, if the fluid storage element had been compressed and expands upon exposure to fluid, the expansion of the fluid storage element would not be affected or inhibited by the attachment or sealing of the flexible panel to the fluid storage element.

The major surface 28 of the flexible panel 12 or panels may be plain, or it can be textured. It is also acceptable in embodiments with multiple panels 12 to have both textured and non-textured panels.

The fluid storage element 14 can be made of any composition known in the art, such as compressed fibrous webs, rolled goods, foam etc. The storage element can be made of any material known in the art such as cotton, rayon, polyester, superabsorbent material, etc.

In one preferred embodiment, the fluid storage element 14 is an absorbent tampon. Absorbent tampons are usually substantially cylindrical masses of compressed absorbent material having a central axis and a radius that defines the outer circumferential surface of the tampon. Such tampons are disclosed in e.g., Haas, U.S. Pat. No. 1,926,900; Dostal, U.S. Pat. No. 3,811,445; Wolff, U.S. Pat. No. 3,422,496; Friese et al., U.S. Pat. No. 6,310,296; Leutwyler et al., U.S. Pat. No. 5,911,712, Truman, U.S. Pat. No. 3,983,875; Agyapong et al., U.S. Pat. No. 6,554,814. Tampons also usually include a fluid-permeable cover (which may include or be replaced by another surface treatment) and a withdrawal string 16 or other removal mechanism.

Absorbent materials useful in the formation of the absorbent body include fiber, foam, superabsorbent, hydrogels, and the like. Preferred absorbent material for the present invention includes foam and fiber. Absorbent foams may include hydrophilic foams, foams that are readily wetted by aqueous fluids as well as foams in which the cell walls that form the foam themselves absorb fluid.

Fibers may be selected from cellulosic fiber, including natural fibers (such as cotton, wood pulp, jute, and the like) and synthetic fibers (such as regenerated cellulose, cellulose nitrate, cellulose acetate, rayon, polyester, polyvinyl alcohol, polyolefin, polyamine, polyamide, polyacrylonitrile, and the like).

The fluid storage element may be substantially enclosed by a fluid-permeable cover 30. The fluid-permeable cover may also enclose the major surfaces 28 of the flexible panel(s) 12. Thus, the cover 30 encloses a majority of the outer surface of the tampon. In addition, either or both ends of the tampon may be enclosed by the cover. Of course, for processing or other reasons, some portions of the surface of the tampon may be free of the cover. For example, the insertion end 22 of the tampon and a portion of the cylindrical surface adjacent this end may be exposed, without the cover to allow the tampon to more readily accept fluids. Additionally, the edges 32 of the flexible panel(s) 12 may also be exposed.

The fluid-permeable cover 30 can ease the insertion of the tampon into the body cavity and can reduce the possibility of fibers being separated from the tampon. Useful covers are known to those of ordinary skill in the art. They may be selected from an outer layer of fibers which are fused together (such as by thermobonding), a nonwoven fabric, an apertured film, or the like. Preferably, the cover has a hydrophobic finish.

Tampons are generally categorized in two classes: applicator tampons and digital tampons, and a certain amount of dimensional stability is useful for each type of tampon. Applicator tampons use a relatively rigid device to contain and protect the tampon prior to use. To insert the tampon into a body cavity, the applicator containing the tampon is partially inserted into the body cavity, and the tampon can be expelled from the applicator into the body cavity. In contrast, digital tampons do not have an applicator to help guide them into the body cavity and require sufficient column strength to allow insertion without using an applicator.

While the applicator tampon is protected by the rigid applicator device and the applicator tampon need not as have as high a degree of column strength as a digital tampon, applicator tampons do require dimensional stability (especially radial) to be acceptable for use. This dimensional stability provides assurance, for example, that the tampon will not prematurely grow and split its packaging material or become wedged in a tampon applicator.

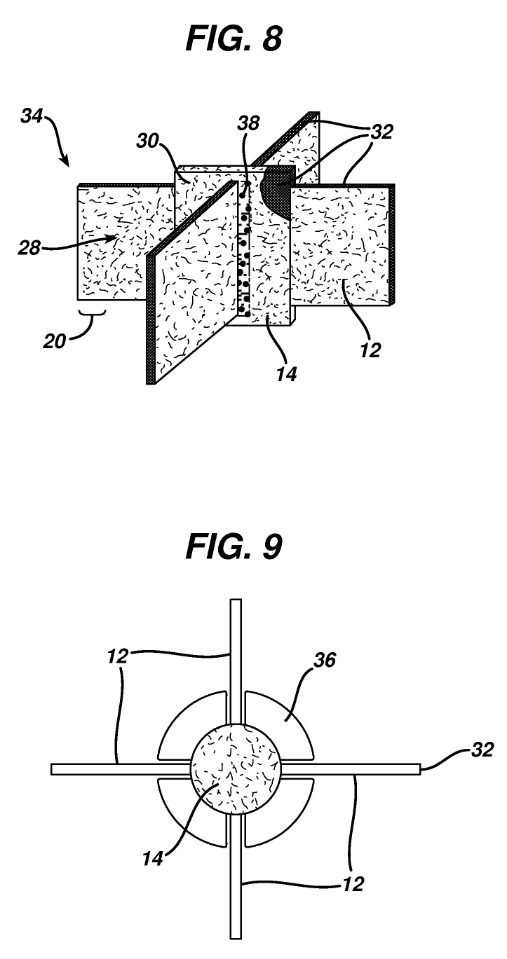

To form a tampon ready for use, an intermediate structure 34 (e.g., as shown in FIG. 8) is typically compressed and heat conditioned in any suitable conventional manner. Pressures and temperatures suitable for this purpose are well known in the art. Typically, the intermediate structure 34 is compressed in both the radial and axial direction using any means well known in the art. While a variety of techniques are known and acceptable for these purposes, a modified tampon compressor machine available from Hauni Machines, Richmond, Va., is suitable. Preferably, the flexible panels 26 are attached to the intermediate structure 34 as shown in FIG. 8.

The intermediate structure 34 may then be compressed to form the fluid storage element 14 as shown in FIG. 9. FIG. 9 shows a series of compression dies 44 provided with narrow axial slits, which allow compression of the fluid storage element 14 without compressing the flexible panels 12. It may also be desirable in some embodiments to attach the flexible panels 12 to the fluid storage element 14 after compression of such element.

The tampon 10 of the present invention may be inserted digitally or through the use of an applicator. If the tampon 10 is to be used for digital insertion, it may be desirable to form the pledget from a layer of absorbent material which has been rolled into a cylindrical shape. Flexible panels 12 could be attached to such a layer in any suitable manner. For example, the attachment tabs 38 shown in FIG. 8 may be used to attach one or more flexible panels 12 to an intermediate structure 34.

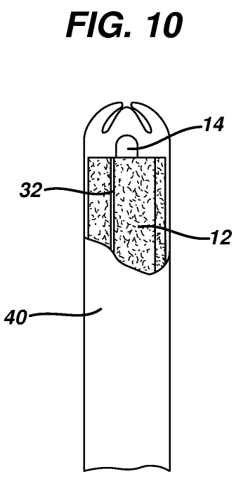

Any of the currently available tampon applicators may also be used for insertion of the tampon of the present invention. Such applicators of typically a “tube and plunger” type arrangement and may be plastic, paper, or other suitable material. Additionally, a “compact” type applicator is also suitable. The flexible nature of the flexible panels 12 allows them to reside in the applicator tube 40 as shown in FIG. 10. The applicator plunger will push the tampon 10 out of the applicator 40 due to the compressed nature of the product. The flexible panels 26 are then available to begin collecting fluid immediately after insertion from their generally uncompressed state.

A withdrawal mechanism, such as withdrawal string 16, is preferably joined to the intravaginal device 10 for removal after use. The withdrawal mechanism is, preferably joined to at least the fluid storage element 14 and extends beyond at least its withdrawal end 24. Any of the withdrawal strings currently known in the art may be used as a suitable withdrawal mechanism, including without limitation, braided (or twisted) cord, yarn, etc. In addition, the withdrawal mechanism can take on other forms such as a ribbon, loop, tab, or the like (including combinations of currently used mechanisms and these other forms). For example, several ribbons may be twisted or braided to provide flexible panels structures.

Further, the intravaginal device can be collapsed for packaging and insertion. For example, at least a portion of a major surface 28 of the flexible panel 12 may be in contact with at least a portion of an outer surface 42 of the fluid storage element 14. This can be achieved by wrapping the flexible panel(s) 12 around the fluid storage element 14, as shown in FIG. 11. Alternatively, the flexible panel(s) 12 may be folded or pleated (e.g., in an accordion-like manner) against the fluid storage element 14, as shown in FIG. 12. The thus-compacted device can then be packaged, (e.g., within an applicator or alone in a wrapper).

The specification and embodiments above are presented to aid in the complete and non-limiting understanding of the invention disclosed herein. Since many variations and embodiments of the invention can be made without departing from its spirit and scope, the invention resides in the claims hereinafter appended.

1-33. (canceled)

34. An intravaginal device comprising
a) a fluid storage element having an insertion end, a withdrawal end, a longitudinal axis and an outer surface; and
b) at least one flexible panel having a distal portion capable of extending radially outward from the fluid storage element; and being bendable about an axis substantially parallel to the longitudinal axis of the fluid storage element;
wherein the at least one flexible panel is in fluid communication with the fluid storage element and the at least one flexible panel encases at least one end of the fluid storage element.

35. The intravaginal device of claim 34, in which the at least one flexible panel encases the insertion end of the fluid storage element.

36. The intravaginal device of claim 34, in which the at least one flexible panel is attached to at least one end of the fluid storage element.

37. The intravaginal device of claim 36, wherein the at least one flexible panel is attached to the intravaginal end of the fluid storage element.

38. The intravaginal device of claim 36, wherein the at least one flexible panel is attached to the outer surface of the fluid storage element.

39. The intravaginal device of claim 34, wherein the at least one flexible panel is attached to itself.

40. The intravaginal device of claim 34, wherein the at least one flexible panel comprises a first flexible panel and a second flexible panel attached to the first flexible panel.

41. The intravaginal device of claim 34, which further comprises a withdrawal string and wherein the at least one flexible panel is attached to the withdrawal string.

42. The intravaginal device of claim 34, wherein the at least one flexible panel is folded against the side surface of the fluid storage element.

43. The intravaginal device of claim 34, wherein the fluid storage element comprises an absorbent structure.

44. The intravaginal device of claim 34, wherein the at least one flexible panel comprises porous medium.

45. The intravaginal device of claim 44, wherein the porous medium comprises a fibrous structure.

46. An elongate, packaged vaginal tampon having a length oriented in an axial direction, comprising:
a) a tampon comprising an absorbent material compressed to a self-sustaining form and having:
i) a fluid storage element having a first end corresponding to an insertion end of the tampon; a second end disposed opposite the first end; a side surface extending between the first end and the second end; and a perimeter, measured in a plane perpendicular to the axial direction; and
ii) at least one flexible panel having a distal portion capable of extending radially outward from the fluid storage element; and being bendable about an axis substantially parallel to the longitudinal axis of the fluid storage element, wherein the at least one flexible panel encases at least one end of the fluid storage element;
b) a packaging element that substantially encloses the tampon with at least a portion of a major surface of the flexible panel in contact with at least a portion of the side surface of the fluid storage element.

47. The packaged tampon of claim 46, wherein the at least one flexible panel has a maximum width that:
a) is measured in a direction perpendicular to its length from the side surface to a most distal portion of the at least one flexible panel as deployed, extending radially outward from the fluid storage element; and
b) exceeds at least 25% of the perimeter, measured at the side surface of the fluid storage element in the region corresponding to the maximum width of the at least one flexible panel.

48. The packaged tampon of claim 46, wherein the flexible panel comprises porous medium.

49. The packaged tampon of claim 48, wherein the porous medium comprises a fibrous structure.

50. A method of providing intravaginal storage of bodily fluids, the method comprising the steps of:
a) removing a vaginal tampon from a package; the tampon having a length oriented in an axial direction and comprising:
i) a fluid storage element that is constructed from an absorbent material compressed to a self-sustaining form and that has a first end corresponding to an insertion end of the tampon, a second end disposed opposite the first end; a side surface extending between the first end and the second end; and a perimeter, measured in a plane perpendicular to the axial direction; and
ii) at least one flexible panel that is extendable radially outward from the fluid storage element and has a length oriented in the axial direction;
wherein the at least one flexible panel encases at least one end of the fluid storage element; and
b) inserting the tampon into a human vagina while maintaining at least a portion of a major surface of the flexible panel in contact with at least a portion of the side surface of the fluid storage element;
whereby the at least one flexible panel is exposed to bodily fluids within the human vagina and transports such bodily fluids toward and around at least a portion of the side surface of the fluid storage element.

51. The method of claim 50, wherein the at least one flexible panel has a maximum width that:
a) is measured in a direction perpendicular to its length from the side surface to a most distal portion of the at least one flexible panel as deployed, extending radially outward from the fluid storage element; and
b) exceeds at least 25% of the perimeter, measured at the side surface of the fluid storage element in the region corresponding to the maximum width of the at least one flexible panel.

52. The method of claim 50, wherein the step of inserting the tampon into a human vagina comprises deploying the tampon from a tampon applicator.
